SEQUENCE LISTING (7 sequences)

SEQ ID NO: 1 - protein, 448 aa, Artificial Sequence
  VARIABLE HEAVY CHAIN REGION OF HUMANIZED 5A8 ANTI-CD4
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVWESNGQPENNYKTTPPVLDSDGSFFLYSRLTVDKSRWQEGNVFSCSVMHEALHNHYTQKSLSLSLGK

SEQ ID NO: 2 - protein, 218 aa, Artificial Sequence
  VARIABLE LIGHT CHAIN REGION OF HUMANIZED 5A8 ANTI-CD4
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 4 - DNA, 37 bp, Artificial Sequence
  OLIGONUCLEOTIDE PRIMER FOR JRCSF ENV ORF
gatcgaattcacgcgtagcagaagacagtggcaatga

SEQ ID NO: 5 - DNA, 37 bp, Artificial Sequence
  OLIGONUCLEOTIDE PRIMER FOR JRCSF ENV ORF
gatcgtcgactctagattttgaccacttgccacccat

SEQ ID NO: 6 - DNA, 29 bp, Artificial Sequence
  OLIGONUCLEOTIDE PRIMER FOR HIV ENV ORF
ggcttaggcatctcctatggcaggaagaa

SEQ ID NO: 7 - DNA, 27 bp, Artificial Sequence
  OLIGONUCLEOTIDE PRIMER FOR HIV ENV ORF
ctgccaatcagggaagtagccttgtgt

(19) United States
(12) Patent Application Publication
(10) Pub. No.: US 2009/0053220 A1
(43) Pub. Date: Feb. 26, 2009

(54) METHODS AND COMPOSITIONS FOR THE INHIBITION OF HIV INFECTION OF T CELLS

(76) Inventors: Thomas Duensing, Katy, TX (US); Sek Chung Fung, Gaithersburg, MD (US); Stanley T. Lewis, Houston, TX (US)

Correspondence Address: JONES DAY, 222 EAST 41ST ST, NEW YORK, NY, 10017, US

(21) Appl. No.: 12/182,808

(22) Filed: Jul. 30, 2008

Related U.S. Application Data
(63) Continuation of application No. PCT/US2007/002991, filed on Feb. 3, 2007.
(60) Provisional application No. 60/837,975, filed on Aug. 16, 2006.
(60) Provisional application No. 60/764,840, filed on Feb. 3, 2006.

(51) Int. Cl. A61K 39/395; A61P 31/18

(52) U.S. Cl. 424/133.1; 424/158.1; 424/173.1; 424/160.1; 424/154.1; 424/145.1

(57) ABSTRACT

The present invention is based upon the surprising discovery that exposure of a non-resistant HIV to a first entry inhibitor, such as an anti-CD4 antibody or a co-receptor inhibitor, which like all current HIV drugs selects for mutations that result in a resistant HIV, surprisingly results in HIV viruses much more susceptible to neutralization by a second entry inhibitor, such as soluble CD4 (sCD4) or an HIV gp41 inhibitor. Therefore, the present invention provides methods and compositions for inhibiting HIV-1 infection in a subject that overcomes the problem of drug resistance.

### BACKGROUND OF THE INVENTION

Acquired immunodeficiency syndrome (“AIDS”) is a disease principally caused by a retrovirus known as the human immunodeficiency virus (“HIV”). HIV weakens the immune system by invading the body and infecting and depleting helper T cells. Helper T cells are essential to a healthy immune system because they control the production of antibodies by B cells, the maturation of cytotoxic T lymphocytes (killer T cells), maturation and activity of macrophages and natural killer cells, and numerous other regulatory and effector functions of the immune system.

Infection by HIV is principally mediated by the viral proteins gp120 and gp41. The gp120 viral protein attaches to the primary receptor CD4 bringing the virus and cell into contact. The extracellular region of CD4 consists of 4 domains (D1, D2, D3, and D4). The HIV-1 gp120 binding site on CD4 comprises amino acids 40 to 60 of CD4 domain 1 (D1). After attachment of gp120 to CD4, gp120 undergoes a conformational change which allows the binding of a chemokine co-receptor (CCR5 or CXCR4). HIV-1 viral isolates from infected patients were originally categorized based on the host cell the virus attached, either a helper T-cell or a macrophage, and thus were designated either T-cell tropic or macrophage tropic. Later, it was determined that this tropism was related to the co-receptor utilized by the virus upon attachment to a cell. Hence, an HIV isolate is now categorized as being either a R5 tropic virus (Binds co-receptor CCR5) or a X4 virus (binds CXCR4). A few HIV viral isolates were found to be dual tropic, i.e., they can bind either co-receptor, and infect either type of host cell.

After the interaction between HIV-1 gp120 and the co-receptor, HIV-1 gp41 is exposed. The gp41 protein then undergoes a harpoon-like conformational change that forms an attachment to the target cell membrane and then uses a spring-like mechanism to form a triple helical, u-shaped protein structure known as the “trimer of hairpins”. Forming the hairpin structure draws the virus to the cell and initiates membrane fusion. This fusion results in the viral particle entering into the target cell and subsequently infecting the cell.

This multi-step process of viral infection requires viral attachment to the CD4 receptor on helper T cells, followed by viral attachment to a co-receptor (typically CXCR4 or CCR5), and viral fusion with the cell. Once inside the cell, the viral RNA is reverse transcribed into DNA, which is then made double stranded for integration into the helper T cell's genome. The inserted viral DNA then uses the host cell's protein translation machinery to transcribe its viral DNA into RNA, translate the viral RNA into viral polyproteins which are then cleaved by viral protease to yield viral proteins used to assemble new viruses. These new viruses ultimately destroy the helper T cell when released. Different drugs and treatment methods have been designed to interfere with one or more of these steps.

Classical treatment methods have targeted primarily the reverse transcription step (reverse transcriptase inhibitors), the protease cleavage step (protease inhibitors), and the viral DNA integration step (integrase inhibitors). Newer approaches have begun to target the viral attachment step (attachment inhibitors), the co-receptor binding step (co-receptor inhibitors), and the fusion step (fusion inhibitors). Attachment inhibitors, co-receptor inhibitors, and fusion inhibitors are collectively referred to as “HIV entry inhibitors”, interfering with viral infection before the virus enters the cell.

Several patents disclose various HIV inhibitors. U.S. Pat. No. 6,309,880 discloses antibodies that target the CD4-binding region of gp120 HIV-1, thus neutralizing HIV-1 before attachment can occur. U.S. Pat. No. 5,871,732 discloses several anti-CD4 antibodies useful for preventing or treating diseases in mammals that target CD4+ lymphocytes. One antibody entry inhibitor, known in the art as 5A8, has been shown to bind to CD4 and block HIV viral entry into the cell.

Attempts to prevent HIV-1 infection using co-receptor inhibitors have also been disclosed. U.S. Application No. 20040209921 discloses heterocyclic compounds that bind to chemokine receptors, including CXCR4 and CCR5, and inhibit HIV infection. U.S. Pat. No. 5,994,515 discloses antibodies that bind to a cellular chemokine receptor protein other than CD4, including CXCR4 and CCR5. U.S. Pat. No. 6,610,834 discloses methods for inhibiting HIV cell entry using antibodies that target CCR5, CCR3, CXCR4, and CCR2B. U.S. Pat. No. 6,528,625 discloses antibodies that bind to mammalian chemokine receptor 5 (CKR-5 or CCR5) and inhibit HIV infection of a cell expressing the receptor.

Several patents disclose compounds that inhibit fusion and therefore viral entry. U.S. Pat. Nos. 6,015,881 and 6,281,331 disclose peptides T-20 and related fusion inhibitor peptides. T-20, also known as Enfuvirtide or FUZEON™, is a 36 amino acid peptide that prevents fusion between HIV-1 and target cells in vitro and in vivo. U.S. Application 20010047080 discloses a protein known as 5-Helix which is designed to inhibit the formation of the trimer-of-hairpins using the N-terminus peptide segment of HIV-1 gp41 to block the C-terminus peptide segment of HIV-1 gp41.

One major problem in HIV treatment is that the virus has a prolific and highly error prone replication process, i.e., does not contain the enzymes needed to correct mistakes made during replication, and the virus reproduces at an extraordinary rate. Replication cycles frequently produce progeny virus with varying degrees of genetic and phenotypic mutations. Moreover, medications used to treat HIV add selection pressure (particularly when the virus is exposed to subtherapeutic levels) such that particular mutant strains thrive whereas susceptible or less hardy strains are inhibited by the medication. These mutant strains are referred to as drug resistant. HIV drug resistance leads to a reduction in the ability of a particular drug or combination of drugs to block HIV replication. For infected patients, this means that HIV drug resistance leads to drugs being less effective or completely ineffective, thus limiting their treatment options.

Resistance typically occurs as a result of mutations in the HIV genetic structure (“RNA”). RNA mutations result in changes in certain proteins, usually enzymes that regulate viral reproduction. HIV relies on many enzymes, e.g., reverse transcriptase, integrase, and protease, to replicate. If a mutation in a single site in the reverse transcriptase gene occurs, the change will remain in the virus as long as it replicates or until another replication error randomly changes it back. Some mutations may cause the virus to become so weak that it cannot replicate effectively. Other mutations may cause the virus to become even more virulent than the original virus.

Current clinical treatments for HIV-1 infections try to address the potential drug resistance by using multiple drugs that target more than one aspect of infection. The standard of care currently is a triple drug combination called Highly Active Antiretroviral Therapy (“HAART”). Current HAART is based upon using potent combinations of drugs, usually three or more drugs from two or more classes. Major forces leading to development of combination therapy for AIDS were the inability of individual drugs (monotherapy) to adequately reduce virus loads and the emergence of drug-resistant mutants, which was usually rapid with any single drug. Viral drug-resistance was considered the major limitation of antiretroviral drugs in the pre-HAART era (Richman, D. D. Antimicrob. Agents Chemother. 37:1207-1213 (1993); D'Aquila, R. T., et al. Ann. Intern. Med. 122:401-408 (1995); Arts, E. J., and M. A. Wainberg. Antimicrob. Agents Chemother. 40:527-540 (1996)). The development of HAART enabled suppression of virus load to undetectable levels for prolonged periods in many patients but has not eliminated problems from viral drug-resistance. The potent combinations used in HAART, when successful, decrease the rate of emergence of resistant variants due to greatly decreased viral load. Nevertheless, treatment failure is usually accompanied by emergence of HIV-1 variants that contain multiple drug-resistance mutations (Fauci, A. S. N. Engl. J. Med. 341:1046-1050 (1999)). In compliant patients, HAART can be effective in reducing mortality and the progression of HIV-1 infection to AIDS. However, many of these drugs are highly toxic and/or require complicated dosing schedules that reduce compliance and limit efficacy.

Because of the nature of HIV infection and the increasing prevalence of drug resistant strains, there is a continuing need for new methods and compositions for preventing infection of target cells by HIV and combating AIDS. In particular, methods that overcome HIV drug resistance are highly desirable.

### SUMMARY OF THE INVENTION

The present invention is based upon the discovery that exposure of a non-resistant HIV to a first entry inhibitor, such as an anti-CD4 antibody, a co-receptor inhibitor, or a fusion inhibitor, which like all current HIV drugs selects for mutations that result in a resistant HIV, surprisingly results in HIV viruses much more susceptible to neutralization by a second entry inhibitor different from the first entry inhibitor administered. Therefore, the present invention provides methods, compositions, and kits for inhibiting HIV-1 infection in a subject that overcomes the problem of drug resistance.

These and other embodiments are achieved by administering to a subject infected by a non-resistant HIV a first HIV entry inhibitor, resulting in resistant HIV or selecting for preexisting resistant HIV, and then exposing the same subject susceptible to infection by the resistant HIV to a second HIV entry inhibitor. The first entry inhibitor induces/selects for mutations that confer resistance. These resistance mutations confer/restore susceptibility or hypersusceptibility to a second HIV entry inhibitor.

One aspect of the invention is a method of inhibiting HIV infection in a subject having HIV/AIDS by administering a first entry inhibitor, e.g., an anti-CD4 antibody, and upon emergence of resistant HIV administering a second entry inhibitor, such as soluble CD4 molecule or a gp120 inhibitor. The method provides for the inhibition of the virus by, e.g., anti-CD4 antibody, for a period of time until resistant viruses begin to appear and then administering a second entry inhibitor, such as sCD4. Alternatively, the method comprises administering an entry inhibitor, e.g., sCD4, for a period of time until resistant viruses begin to appear and then administering an anti-CD4 antibody. Another aspect of the invention is an alternating treatment approach, wherein the subject is treated with a first entry inhibitor for a period of time until resistant viruses emerge and then a second entry inhibitor is administered. When resistant viruses emerge that are resistant to the second inhibitor, the first entry inhibitor is again administered. This alternating treatment can be continued as long as the virus responds.

One embodiment of a sCD4 molecule useful in the present invention comprises at least the binding region for HIV comprising the sequence of Domain 1 from AA25 to AA123 of SEQ ID NO 3. (FIG. 3) Another embodiment comprises sCD4 having the sequence of SEQ ID NO 3.

Another aspect of the invention is a method of administering anti-CD4 antibody and a variant sCD4 simultaneously. The wild-type sCD4 molecule comprises four domains, domain 1 being the site of viral attachment (SEQ ID NO 3). The variant sCD4 molecule to be administered in the present invention comprises one or more of the four domains but lacks the binding site recognized by the anti-CD4 antibody to be administered. Alternatively, the variant sCD4 comprises a mutation in the binding site recognized by the anti-CD4 antibody or a substitution in the binding site. This mutation, deletion, or substitution prevents binding of the antibody to the variant sCD4 molecule to be administered, but still allows binding of the HIV virus present in the subject to be treated.

Thus, the invention includes a composition comprising an anti-CD4 antibody and a variant sCD4 molecule containing a mutation, deletion, or substitution in the binding site recognized by the anti-CD4 antibody. The variant sCD4 molecule comprises: (1) the entire sCD4 molecule with a mutation in the binding site recognized by the anti-CD4 antibody to be used; (2) the entire sCD4 molecule having a deletion in the binding site recognized by the anti-CD4 antibody to be used; (3) the entire sCD4 molecule having a substitution in the binding site recognized by the anti-CD4 antibody to be used; or (4) a sCD4 molecule comprising at least the binding region of HIV but lacking the binding site recognized by the anti-CD4 antibody to be used. The variant sCD4 molecule may also comprise a peptide that extends the half-life of the sCD4 molecule, such as an Fc fusion protein.

The anti-CD4 antibody comprises any antibody molecule or antibody fragment thereof that specifically binds the extracellular domain of the CD4 receptor and is non-CD4 depleting. This antibody may be monoclonal, chimeric, humanized, human, or a single chain antibody or domain antibody. The antibody fragment may be a Fab, Fab′, or F(ab′)₂. One embodiment of an anti-CD4 antibody is 5A8 disclosed in U.S. Pat. No. 5,871,732 (incorporated herein by reference) and may be produced from the hybridoma having accession number HB 10881. Another embodiment of an anti-CD4 antibody useful in the present invention comprises a humanized recombinant antibody, wherein the heavy chain variable region comprises SEQ ID NO 1 and the light chain variable region comprises SEQ ID NO 2. Another embodiment of an anti-CD4 antibody useful in the present invention comprises an antibody the following CDRs: (a) light chain CDR1 is AA24-AA40 of SEQ ID NO: 2; (b) light chain CDR2 is AA56-AA62 of SEQ ID NO: 2; (c) light chain CDR3 is AA95-AA102 of SEQ ID NO: 2, (d) heavy chain CDR1 is AA26-AA35 of SEQ ID NO: 1; (e) heavy chain CDR2 is AA50-AA66 of SEQ ID NO: 1; and (f) heavy chain CDR3 is AA99-AA111 of SEQ ID NO: 1.

Other entry inhibitors may include BMS-488,043 (a small molecule that specifically and reversibly binds to HIV gp120 and prevents the attachment of HIV to CD4+ T-lymphocytes), PRO-542 (a soluble antibody-like fusion protein that prevents attachment of HIV to CD4+ T-lymphocytes by binding to gp120), and antibodies targeting gp120, such as those disclosed in U.S. Pat. No. 5,854,400 or U.S. Pat. No. 5,981,278.

Other entry inhibitors may include co-receptor inhibitors that are polypeptides or other compounds, including antibodies that interact with co-receptors to inhibit or prevent co-receptor interaction with its ligand, e.g., gp120. Co-receptor inhibitors useful in the present invention include, e.g., Sch-D, GW-873,140, UK-427,857, PRO-140 (CCR5-receptor inhibitors), and AMD-070, (a CXCR4 receptor inhibitor).

Other entry inhibitors may include fusion inhibitors that are polypeptides or other compounds, including antibodies that interact with, e.g., gp41, to inhibit or prevent fusion of HIV and the cell to be infected. One example of a fusion inhibitor useful in the present invention is T-20.

These and other aspects of the invention will be apparent to those skilled in the art upon consideration of the following detailed description.

### BRIEF DESCRIPTION OF THE FIGURES

FIG. 1 depicts the proposed open and closed HIV-1 viral configurations.

FIG. 2 depicts the variable sequence of the heavy and light chains of humanized 5A8. The CDRs are underlined.

FIG. 3 depicts the sequence of soluble CD4, domain 1 is indicated by Bold, underline.

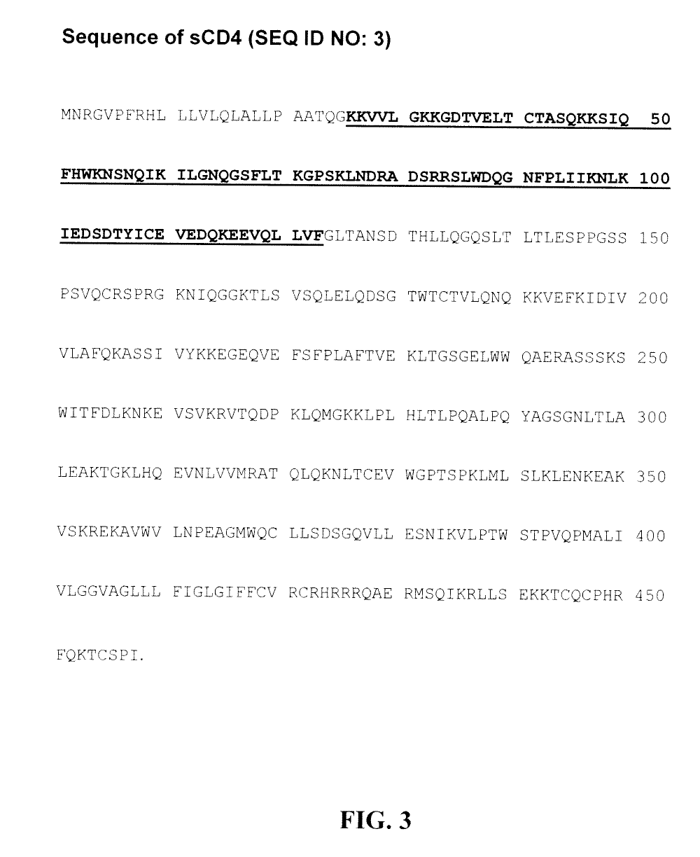

FIG. 4 depicts the inverse susceptibility of HIV to 5A8 and sCD4.

### DEFINITIONS

Terms used throughout this application are to be construed with ordinary and typical meaning to those of ordinary skill in the art. However, Applicants desire that the following terms be given the particular definition as defined below.

The term “entry inhibitor(s)” means an attachment inhibitor(s), CD4-gp120 interaction inhibitor(s), or a co-receptor inhibitor(s), and their functionally equivalent peptides or functionally equivalent compounds, as appropriate, either collectively or individually.

The term “non-resistant HIV” refers to an HIV that lacks known resistance to the drug to be administered.

The term “resistant HIV” means an HIV (1) that is produced when a “non-resistant HIV” is exposed to a chosen drug and mutates resulting in an HIV that is resistant to that drug, or (2) that requires more than the recommended dose of the drug to suppress the HIV infection.

The term “subject” means a primate having an HIV infection. The primate treated according to the present invention may be a human.

The term “functionally equivalent binding peptides” means a fragment of a polypeptide that has the same biological activity as the polypeptide.

The term “antibody” is used in the broadest sense and specifically covers non-native sequence antibodies, monoclonal antibodies, antibody compositions with polyepitopic specificity, bispecific antibodies, diabodies, single-domain antibodies and single-chain molecules.

“Antibody fragments” comprise a portion of an intact antibody comprising the antigen-binding or variable region thereof. Examples of antibody fragments include Fab, Fab′, F(ab′)₂, and Fv fragments; diabodies; linear antibodies; single-chain antibody molecules; and multispecific antibodies formed from antibody fragment(s).

An “intact” antibody is one which comprises an antigen-binding variable region as well as a light chain constant domain (C_{L}) and heavy chain constant domains, C_{H}1, C_{H}2 and C_{H}3. The constant domains may be native sequence constant domains (e.g. human native sequence constant domains) or amino acid sequence variant thereof. The intact antibody may have one or more effector functions.

Antibody “effector functions” refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc.

### DETAILED DESCRIPTION

This invention is not limited to the particular methodology, protocols, cell lines, vectors, or reagents described herein because they may vary. Further, the terminology used herein is for the purpose of describing particular embodiments only and is not intended to limit the scope of the present invention. As used herein and in the appended claims, the singular forms “a”, “an”, and “the” include plural reference unless the context clearly dictates otherwise, e.g., reference to “a host cell” includes a plurality of such host cells.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

All patents and publications mentioned herein are incorporated herein by reference to the extent allowed by law for the purpose of describing and disclosing the proteins enzymes, vectors, host cells, and methodologies reported therein that might be used with the present invention. However, nothing herein is to be construed as an admission that the invention is not entitled to antedate such disclosure by virtue of prior invention.

### HIV Infection and Resistance

Infection by HIV is principally mediated by the viral proteins gp120 and gp41. The gp120 viral protein attaches to the primary receptor CD4 bringing the virus and cell into contact. The extracellular region of CD4 consists of 4 domains (D1, D2, D3, and D4). The HIV-1 gp120 binding site on CD4 comprises amino acids 40 to 60 of CD4 domain 1 (D1). After attachment of gp120 to CD4, gp120 undergoes a conformational change which allows the binding of a chemokine co-receptor (CCR5 or CXCR4). HIV-1 viral isolates from infected patients were originally categorized based on the host cell the virus attached, either a helper T-cell or a macrophage, and thus were designated either T-cell tropic or macrophage tropic. Later, it was determined that this tropism was related to the co-receptor utilized by the virus upon attachment to a cell. Hence, an HIV isolate is now categorized as being either a R5 tropic virus (Binds co-receptor CCR5) or a XR4 virus (binds CXCR4). A few HIV viral isolates were found to be dual tropic, i.e., they can bind either co-receptor, and infect either type of host cell. CXCR4 is the primary co-receptor used by most primary HIV-1 isolates. CCR5 is the co-receptor used by macrophage-tropic primary HIV-1 isolates.

After the interaction between HIV-1 gp120 and the co-receptor, HIV-1 gp41 is exposed. The gp41 protein then undergoes a harpoon-like conformational change that forms an attachment to the target cell membrane and then uses a spring-like mechanism to form a triple helical, u-shaped protein structure known as the “trimer of hairpins”. Forming the hairpin structure draws the virus to the cell and initiates membrane fusion. This fusion results in the viral particle entering into the target cell and subsequently infecting the cell.

This multi-step process of viral infection requires viral attachment to the CD4 receptor on helper T cells, followed by viral attachment to a co-receptor (typically CXCR4 or CCR5), and viral fusion with the cell. Once inside the cell, the viral RNA is reverse transcribed into DNA, which is then made double stranded for integration into the helper T cell's genome. The inserted viral DNA then uses the host cell's protein translation machinery to transcribe its viral DNA into RNA, translate the viral RNA into viral polyproteins which are then cleaved by viral protease to yield viral proteins used to assemble new viruses. These new viruses ultimately destroy the helper T cell when released. Different drugs and treatment methods have been designed to interfere with one or more of these steps.

Classical treatment methods have targeted primarily the reverse transcription step (reverse transcriptase inhibitors), the protease cleavage step (protease inhibitors), and the viral DNA integration step (integrase inhibitors). Newer approaches have begun to target the viral attachment step (attachment inhibitors), the co-receptor binding step (co-receptor inhibitors), and the fusion step (fusion inhibitors). Attachment inhibitors, co-receptor inhibitors, and fusion inhibitors are collectively referred to as “HIV entry inhibitors”, interfering with viral infection before the virus enters the cell.

One major problem in HIV treatment is that the virus has a prolific and highly error prone replication process, i.e., does not contain the enzymes needed to correct mistakes made during replication, and the virus reproduces at an extraordinary rate. Replication cycles frequently produce progeny virus with varying degrees of genetic and phenotypic mutations. Moreover, medications used to treat HIV add selection pressure (particularly when the virus is exposed to subtherapeutic levels) such that particular mutant strains thrive whereas susceptible or less hardy strains are inhibited by the medication. These mutant strains are referred to as drug resistant. HIV drug resistance leads to a reduction in the ability of a particular drug or combination of drugs to block HIV replication. For infected patients, this means that HIV drug resistance leads to drugs being less effective or completely ineffective, thus limiting their treatment options.

Resistance typically occurs as a result of mutations in the HIV genetic structure (“RNA”). RNA mutations result in changes in certain proteins, usually enzymes that regulate viral reproduction. HIV relies on many enzymes, e.g., reverse transcriptase, integrase, and protease, to replicate. If a mutation in a single site in the reverse transcriptase gene occurs, the change will remain in the virus as long as it replicates or until another replication error randomly changes it back. Some mutations may cause the virus to become so weak that it cannot replicate effectively. Other mutations may cause the virus to become even more virulent than the original virus.

Several important features of the HIV-1 envelope proteins must be considered in approaches for development of antiviral drugs. First, the high degree of sequence variability of HIV-1 ENV presents a significant challenge for antiviral drug or vaccine development. Diverse HIV-1 isolates have been classified into subtypes A through K (major group, M), as well as the highly divergent groups N and O (outlier) by comparison of amino acid sequences in ENV or gag regions (Robertson, D. L., et al. Science 288:55-56 (2000)). Variability in ENV is the basis for much of the differences between subtypes of HIV-1. Envelope variability also governs HIV-1 co-receptor usage and cell tropism. The high variability of ENV, particularly in gp120, is also important in the evasion of antiviral immune responses (Klenerman, P., et al. Curr. Opin. Microbiol. 5:408-413 (2002); Wyatt, R., and J. Sodroski. Science 280:1884-1888 (1998)). Drugs that target more conserved regions and that are active against all or most subtypes will be much more useful than subtype-specific inhibitors of HIV-1.

There are also major differences in ENV between primary isolates of HIV-1 and laboratory strains. Most notable have been the differences between primary isolates and lab strains in susceptibility to neutralizing antibodies (Burton, D. R., et al. Science 265:1024-1027 (1994)); Sullivan, N., et al. J. Virol. 69:4413-4422 (1995)). Therefore, it is important to include drug screens with several strains of HIV-1, including primary isolates, in the strategy for development of antiviral agents that interact with an envelope glycoprotein.

The present invention is based upon the surprising discovery that exposure of a non-resistant HIV to a first entry inhibitor, such as an anti-CD4 antibody, results in selective mutations that produce a resistant HIV that is surprisingly more susceptible to neutralization by a second entry inhibitor, such as sCD4 or an anti-gp120 antibody. Thus, in one aspect, the present invention provides a new and novel method for inhibiting HIV infection using sequential or simultaneous administration of two or more entry inhibitor HIV drugs. In one particular embodiment of the invention, the method comprises exposing a subject having susceptible non-resistant HIV molecules to a first entry inhibitor, such as an anti-CD4 antibody or a co-receptor inhibitor. Subsequent to exposure to the first entry inhibitor, HIV develops resistance or selects for preexisting resistant HIV within the subject. Resistance to, e.g., an anti-CD4 antibody confers susceptibility or hyper-susceptibility not originally present or to the same degree to a certain amount of a second entry inhibitor, e.g., sCD4. More specifically, the method comprises administering an amount of anti-CD4 antibody sufficient to reduce the viral load in a subject in need of such treatment, and upon emergence of resistant HIV, administering an amount of sCD4 sufficient to reduce the viral load of HIV resistant to the anti-CD4 antibody.

Without being bound by any theory, it is believed the mutations required to produce the resistant HIV occur in HIV epitopes that make the resistant HIV highly susceptible to inhibition by a second inhibitor, such as sCD4. The mutation(s) required to confer resistance to anti-CD4 antibody change the conformation of envelope or other proteins (e.g., gp120) exposing epitopes that are ordinarily hidden from a soluble CD4 molecule. The exposure of these hidden epitopes makes the HIV resistant to an anti-CD4 antibody, but highly susceptible to sCD4.

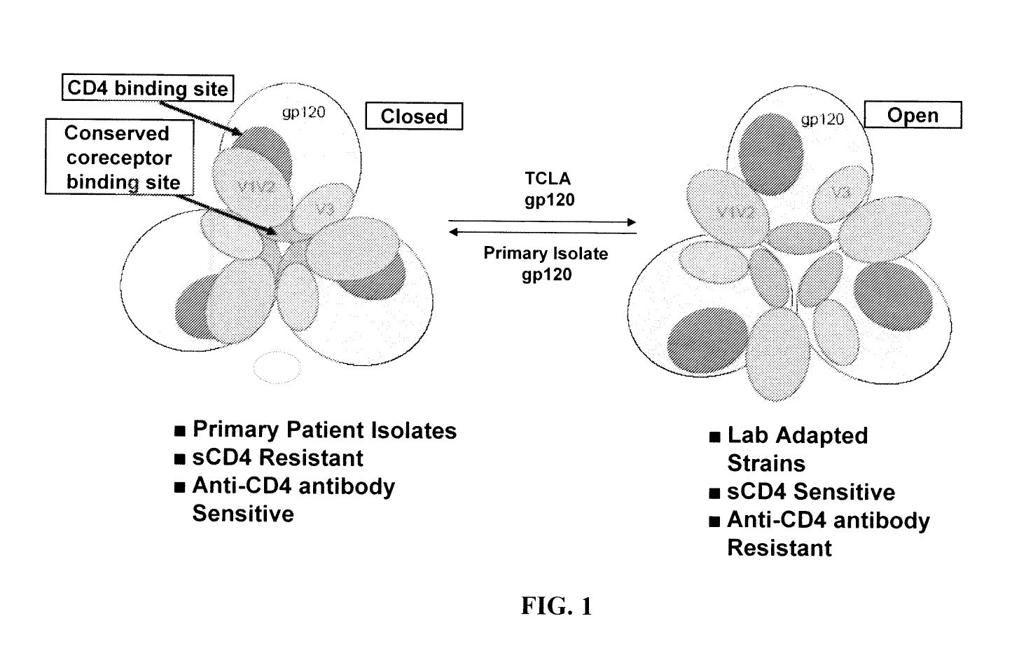

This theory is based on the observation that HIV has two conformations (depicted in FIG. 1), a closed conformation which the inventors have observed to be resistant to sCD4 and sensitive to anti-CD4 antibody, and an open conformation that is sensitive to sCD4 and resistant to anti-CD4 antibody. Surprisingly, virus that are in the closed confirmation more closely resemble virus known as primary isolates, i.e. isolated from patients. Virus that are in the open confirmation more closely resemble virus known as lab-adapted strains. The inventors postulate that the virus isolated from patients has adopted the closed confirmation to avoid the host's immune system, whereas the laboratory strains are not being attacked by the immune system and adopt a more open conformation.

In an alternative embodiment of the present invention, an anti-CD4 antibody and a variant sCD4 molecule are be administered simultaneously, and as the virus becomes resistant to one, it becomes susceptible to the other. The variant sCD4 molecule to be administered in the present invention comprises one or more of the four domains, but lacks the binding site recognized by the anti-CD4 antibody to be administered. Alternatively, the variant sCD4 comprises a mutation in the binding site recognized by the anti-CD4 antibody or a substitution in the binding site. This mutation, deletion, or substitution prevents binding of the antibody to the sCD4 molecule to be administered, but still allows binding of the HIV virus present in the subject to be treated.

Thus, the invention includes a composition comprising an anti-CD4 antibody and a variant sCD4 molecule containing a mutation, deletion, or substitution in the binding site recognized by the anti-CD4 antibody. The variant sCD4 molecule comprises: (1) the entire sCD4 molecule with a mutation in the binding site recognized by the anti-CD4 antibody to be used; (2) the entire sCD4 molecule having a deletion in the binding site recognized by the anti-CD4 antibody to be used; (3) the entire sCD4 molecule having a substitution in the binding site recognized by the anti-CD4 antibody to be used; or (4) a sCD4 molecule comprising at least the binding region of HIV but lacking the binding site recognized by the anti-CD4 antibody to be used. The sCD4 molecule may also comprise an immunoglobulin constant region fragment that extends the half-life of the sCD4 molecule, such as an IgG Fc region.

Another aspect of the invention is a kit or article of manufacture comprising two vials, one vial containing the anti-CD4 antibody and the second vial containing a variant sCD4 molecule. Alternatively, the kit or article of manufacture may comprise a single vial containing a composition composed of the anti-CD4 antibody and a variant sCD4 molecule. Typically, the kit contains amounts sufficient to administer one or more doses for inhibition of the HIV infection. A typical dosage might range from about 1 μg/kg to up to 100 mg/kg of patient body weight or more per day, preferably about 10 μg/kg/day to 10 mg/kg/day. Amounts of other drugs to include in the kit are determined by reference to approved or recommended dosages for the particular drug. The kit may contain humanized 5A8 antibody and a variant sCD4 molecule. Optionally, the kit may also contain a non-entry inhibitor anti-HIV drug such as integrase inhibitors, nucleoside reverse transcriptase inhibitors, non-nucleoside reverse transcriptase inhibitors, and HIV protease inhibitors.

One embodiment of the present invention includes monoclonal antibodies that bind to CD4. Another embodiment includes antibodies that bind CD4 and permit attachment of gp120 but inhibit or prevent HIV-1 infection, including, but not limited to, the antibodies disclosed in U.S. Pat. No. 5,871,732. Known anti-CD4 antibodies useful in the present invention include 5A8 (an anti-CD4 monoclonal antibody that permits HIV to bind to CD4 but prevents entry of HIV by binding to the CD4 receptor on the cell's surface and thereby inhibiting infection).

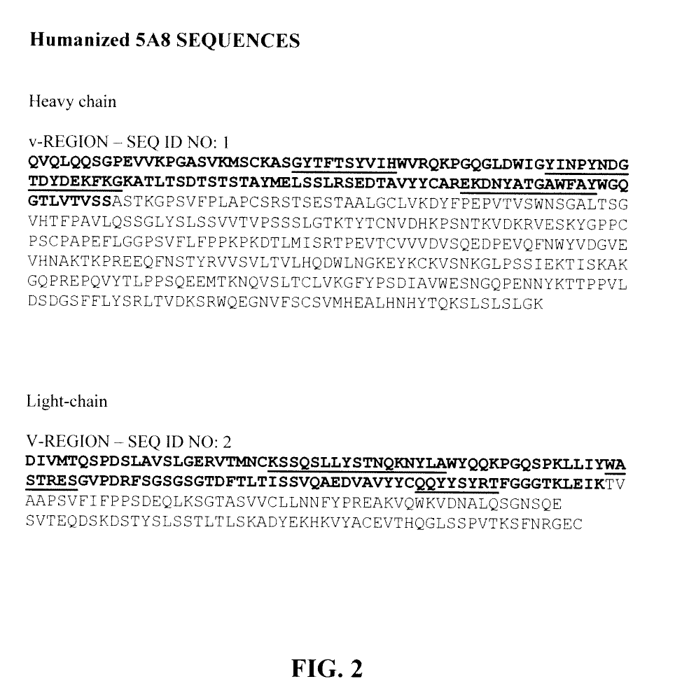

Experiments have shown that treating non-resistant HIV strains with humanized 5A8 consistently produces resistant HIV strains that are highly susceptible to treatment with sCD4. Humanized 5A8 containing an IgG4 MAb constant region inhibits HIV-1 entry by binding to the extracellular domain 2 of CD4 and preventing post-binding entry of the virus into CD4+ cells. Because the antibody binding site on CD4 is distinct from the site required for the binding of HIV-1 envelope gp120, 5A8 permits the binding of gp120 to the CD4 receptor but inhibits HIV-1 entry into these CD4+ T-cells. The sequence of humanized 5A8 is depicted in FIG. 2.

In another aspect, the present invention provides a method for overcoming HIV drug resistance created by treating a subject infected with HIV with an anti-CD4 antibody in combination with treatment with sCD4, either sequentially or simultaneously.

In a further aspect, the present invention provides compositions useful for preventing and treating infection by HIV. One embodiment of the present invention is a composition comprising an anti-CD4 antibody alone or in combination with a pharmaceutically acceptable carrier, such as various carriers, adjuvants, additives, and diluents. Another embodiment of the invention is a composition comprising a soluble CD4 molecule alone or in combination with a pharmaceutically acceptable carrier such as stabilizers, adjuvants, additives, and diluents. Another embodiment of the invention is a composition comprising: (1) an anti-CD4 antibody, (2) a sCD4 molecule comprising at least the binding site for HIV gp120 but lacking the binding site recognized by the anti-CD4 antibody of (1) above; and optionally (3) a pharmaceutically acceptable carrier such as stabilizers, adjuvants, additives, and diluents. Any of these compositions when used to treat an HIV infection may be used in combination with any other HIV therapy.

The entry inhibitors of the present invention include small molecules, peptides, and polypeptides, including antibodies, and their functionally equivalent peptides and functionally equivalent compounds. The entry inhibitors also include antibodies generated by an animal in response to an antigen administered to the animal, e.g., a vaccine containing an antigen that induces the body to generate an entry inhibitor, preferably a second entry inhibitor in accordance with the present invention.

The entry inhibitors of the present invention comprise peptides, CD4-gp120 interaction inhibitors that are polypeptides or other compounds that, e.g., bind to the CD4 receptor on target cells inhibiting or preventing HIV-1 attachment to the target cells or that bind to a viral protein on HIV-1 thereby inhibiting or preventing attachment or cellular fusion between HIV-1 and the target cells. Generally, these entryCD4-gp120 interaction inhibitors are antibodies, antibody fragments, CD4 inhibitors, such as gp120, or gp120 inhibitors comprising a fragment of CD4 (or CD4 variants) such as a fusion protein of CD4 with human IgG2.

Entry inhibitors may be polyclonal or monoclonal antibodies that bind to gp120 and prevent attachment of gp120 to CD4 or may permit attachment of gp120 to CD4 but inhibit or prevent fusion of the virus and the target cell. These inhibitors may also be polyclonal or monoclonal antibodies that bind to CD4 and prevent attachment of gp120 to CD4 or permit attachment of gp120 to CD4 but inhibit or prevent fusion of the virus and the target cell.

One embodiment of the present invention includes monoclonal antibodies that bind to CD4 and permit attachment of gp120 but inhibit or prevent fusion of HIV-1 and the target cell, including, but not limited to, the antibodies disclosed in U.S. Pat. No. 5,871,732. Known CD4-gp120 interaction inhibitors useful in the present invention include 5A8 (an anti-CD4 monoclonal antibody that permits HIV to bind to CD4 but prevents entry of HIV into the target cell by binding to the CD4 receptor on the cell's surface and thereby blocking infection), BMS-488,043 (a CD4-gp120 interaction small molecule that specifically and reversibly binds to HIV gp120 and prevents the attachment of HIV to CD4+ T-lymphocytes), PRO-542 (a soluble antibody-like fusion protein that prevents attachment of HIV to CD4+ T-lymphocytes by binding to gp120), soluble CD4 and its variants, and antibodies targeting gp120 or different domains of CD4.

The entry inhibitors of the present invention also comprise co-receptor inhibitors that are polypeptides or other compounds that interact with target cell co-receptors to inhibit or prevent co-receptor interaction with its ligand, e.g., gp120. Co-receptor inhibitors useful in the present invention include, e.g., Sch-D, GW-873,140, UK-427,857, PRO-140 (CCR5-receptor inhibitors), and AMD-070, (a CXCR4 receptor inhibitor).

In several embodiments, the method comprises exposing target cells to a first entry inhibitor comprising an attachment inhibitor, a CD4-gp120 interaction inhibitor, or a co-receptor inhibitor, and a second entry inhibitor comprising an attachment inhibitor, a CD4-gp120 interaction inhibitor, or a co-receptor inhibitor. These entry inhibitors can be used in various combinations in the present method, e.g., coreceptor inhibitor as the first entry inhibitor and a CD4-gp120 interaction inhibitor as the second entry inhibitor or an attachment inhibitor as the first entry inhibitor and a CD4-gp120 interaction as the second entry inhibitor. The second entry inhibitor is an entry inhibitor other than the first entry inhibitor, i.e., the first and second entry inhibitors are not the same entry inhibitor. The most applicable method for a particular treatment scheme can be selected by the skilled artisan based upon the effectiveness and other characteristics of the entry inhibitors. Obviously, entry inhibitors that adversely interact with each other must be used sequentially and not in conjunction, e.g., an antibody and its isolated receptor.

One embodiment of the present invention is a method comprising exposing target cells to a first entry inhibitor comprising a CD4-gp120 interaction inhibitor and a second entry inhibitor comprising a different CD4-gp120 interaction inhibitor. The first CD4-gp120 interaction inhibitor is, e.g., an anti-CD4 antibody, such as 5A8, and the second CD4-gp120 interaction inhibitor is, e.g., BMS-488,043, PRO-542, anti-CD4 antibodies, or sCD4 or its variants.

In a further embodiment, the method comprises administering to a subject a first entry inhibitor comprising an CD4-gp120 interaction inhibitor and a second entry inhibitor comprising a co-receptor inhibitor. The first CD4-gp120 interaction inhibitor is, e.g. an anti-CD4 antibody, such as 5A8, and the second CD4-gp120 interaction inhibitor is e.g. Sch-D, GW-873,140, UK-427,857, PRO-140 (CCR5-receptor inhibitors), or AMD-070, (CXCR4 receptor inhibitor).

In another embodiment, the method comprises administering to a subject a first entry inhibitor comprising an CD4-gp120 interaction inhibitor and a second entry inhibitor comprising a fusion inhibitor. The first CD4-gp120 interaction inhibitor is an anti-CD4 antibody, such as 5A8, and the second entry inhibitor is e.g., an anti-gp120 antibody.

In another embodiment, the method further comprises administering to a subject entry inhibitors of the present invention in combination with at least one other non-entry inhibitor anti-HIV drug, such as an integrase inhibitor, a nucleoside reverse transcriptase inhibitor, a non-nucleoside reverse transcriptase inhibitors, or a HIV protease inhibitor, including in HAART therapy like treatments. Preferably the drug is at least one integrase inhibitor, and/or at least one transcriptase inhibitor, and/or at least one protease inhibitor. Such methods are useful in HAART regimens. In one embodiment, the method comprises administering at least one entry inhibitor and one or more integrase, transcriptase, or protease inhibitors.

The compounds and compositions of the present invention can be administered or co-administered to a patient by any suitable method known in the art, particularly for administering peptides, polypeptides, or antibodies. Such methods include, but are not limited to, injections, implants, and the like. Injections are preferred because they permit precise control of the timing and dosage levels used for administration. The compounds and compositions of the present invention can be administered parenterally, intraperitoneally, intravenously, intraarterially, transdermally, sublingually, intramuscularly, subcutaneously, intraarticularly, or intrathecally.

In one embodiment, the first HIV entry inhibitor and the second HIV entry inhibitor are administered sequentially. In this administration method, the first HIV entry inhibitor is administered alone until viral resistance is suspected or detected. Then, the second HIV entry inhibitor is administered, alone or in combination with the first entry inhibitor, to prevent infection by the resistant HIV. Being administered “alone” refers only to the entry inhibitors. The entry inhibitors can be administered in combination with any other current HIV therapy regimen the subject may already be receiving.

The entry inhibitors can be administered in a single dose or can be administered in multiple doses over a defined period. For example, one of the entry inhibitors can be administered by intravenous injection as a single dose and the other entry inhibitor can be administered by daily injection or orally over a period of several days. Many such administration patterns will be apparent to those skilled in the art.

The amount or dosage of entry inhibitors administered may vary depending upon the entry inhibitor, the age of the subject, size of the subject, health of the subject, the administration pattern, the severity of the disease, and whether the dose is to act therapeutically or prophylactically. Generally, entry inhibitors are administered to the subject in dosages of from about 1 to 50 milligrams per kilogram of body weight (mg/kg), preferably from about 5 to 30 mg/kg. The entry inhibitors are typically administered on a weekly schedule but may be administered on a bi-weekly or monthly schedule. For repeated administrations over several days, weeks, or longer, depending on the condition, the treatment is repeated until a desired suppression of HIV viral load and/or disease symptoms occurs or the desired improvement in the subject's condition is achieved. The dosage may be readministered at intervals ranging from once a week to once every six months. The determination of the optimum dosage and of optimum route and frequency of administration is well within the knowledge of those skilled in the art. Similarly, dosages for other drugs within the scope of the present invention can be determined without undue experimentation.

The compositions of the present invention include pharmaceutically acceptable carriers that are inherently nontoxic and non-therapeutic. “Pharmaceutically acceptable” carriers, excipients, or stabilizers are ones which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins, chelating agents such as EDTA; sugar alcohols such as mannitol or sorbitol; salt-forming counterions such as sodium; and/or nonionic surfactants such as TWEEN™, polyethylene glycol (PEG), and PLURONICS™. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. Such pharmaceutical compositions may be prepared and formulated in dosage forms by methods known in the art.

In another aspect, the present invention provides a means for communicating information about or instructions for using first and second entry inhibitors to prevent infection of target cells by HIV and to prevent or treat HIV infection. The communicating means comprises a document or visual display that contains the information or instructions. Preferably, the communication is a web site displayed on a visual monitor, brochure, or package insert containing such information or instructions.

Useful information includes the fact that the entry inhibitors work in combination according to the present invention, details about the side effects, if any, caused by using the entry inhibitors in combination and in combination with other drugs, and contact information for patients to use if they have a question about the entry inhibitors or their use. Useful instructions include entry inhibitor dosages, administration amounts and frequency, and administration routes. The communication means is useful for instructing a patient on the benefits of using the entry inhibitors of the present invention and communicating the approved methods for administering the inhibitors to a patient.

### Generation of Antibodies

The antibodies of the present invention may be generated by any suitable method known in the art. The antibodies of the present invention may comprise polyclonal antibodies. Methods of preparing polyclonal antibodies are known to the skilled artisan (Harrow, et al., Antibodies: a Laboratory Manual, (Cold spring Harbor Laboratory Press, 2nd ed. (1988), which is hereby incorporated herein by reference in its entirety).

For example, an immunogen, such as sCD4 or HIV gp120, may be administered to various host animals including, but not limited to, rabbits, mice, rats, etc., to induce the production of sera containing polyclonal antibodies specific for the antigen. The administration of the immunogen may entail one or more injections of an immunizing agent and, if desired, an adjuvant. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BOG (bacille Calmette-Guerin) and Corynebacterium parvum. Additional examples of adjuvants which may be employed include the MPL-TDM adjuvant (monophosphoryl lipid A, synthetic trehalose dicorynomycolate).

Immunization protocols are well known in the art in the art and may be performed by any method that elicits an immune response in the animal host chosen. Adjuvants are also well known in the art. Typically, the immunogen (with or without adjuvant) is injected into the mammal by multiple subcutaneous or intraperitoneal injections, or intramuscularly or through IV. The immunogen may include, e.g., a CD-4 polypeptide, a fusion protein or variants thereof. Depending upon the nature of the polypeptides (i.e., percent hydrophobicity, percent hydrophilicity, stability, net charge, isoelectric point etc.), it may be useful to conjugate the immunogen to a protein known to be immunogenic in the mammal being immunized. Such conjugation includes either chemical conjugation by derivatizing active chemical functional groups to both the immunogen and the immunogenic protein to be conjugated such that a covalent bond is formed, or through fusion-protein based methodology, or other methods known to the skilled artisan. Examples of such immunogenic proteins include, but are not limited to, keyhole limpet hemocyanin, ovalbumin, serum albumin, bovine thyroglobulin, soybean trypsin inhibitor, and promiscuous T helper peptides. Various adjuvants may be used to increase the immunological response as described above.

The antibodies of the present invention comprise monoclonal antibodies. Monoclonal antibodies may be prepared using hybridoma technology, such as those described by Kohler and Milstein, Nature, 256:495 (1975) and U.S. Pat. No. 4,376,110, by Harlow, et al., Antibodies: A Laboratory Manual, (Cold spring Harbor Laboratory Press, 2nd ed. (1988), by Hammerling, et al., Monoclonal Antibodies and T-Cell Hybridomas (Elsevier, N.Y., (1981)), or other methods known to the artisan. Other examples of methods which may be employed for producing monoclonal antibodies include, but are not limited to, the human B-cell hybridoma technique (Kosbor et al., 1983, Immunology Today 4:72, Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the MAb of this invention may be cultivated in vitro or in vivo.

Using typical hybridoma techniques, a host such as a mouse, a humanized mouse, a mouse with a human immune system, hamster, rabbit, camel or any other appropriate host animal, is typically immunized with an immunogen to elicit lymphocytes that produce or are capable of producing antibodies that will specifically bind to the target. Alternatively, lymphocytes may be immunized in vitro with the antigen.

Generally, in making antibody-producing hybridomas, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986), pp. 59-103). Immortalized cell lines are usually transformed mammalian cells, particularly myeloma cells of rodent, bovine or human origin. Typically, a rat or mouse myeloma cell line is employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), substances that prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines may also be used for the production of human monoclonal antibodies (Kozbor, J. Immunol., 133:3001 (1984); Brodeur et al., Monoclonal Antibody Production Techniques and Applications, Marcel Dekker. Inc., New York, (1987) pp. 51-63).

The culture medium in which the hybridoma cells are cultured can then be assayed for the presence of monoclonal antibodies directed against the target, such as CD4 receptor. The binding specificity of monoclonal antibodies produced by the hybridoma cells is determined by, e.g., immunoprecipitation or by an in vitro binding assay, such as radioimmunoassay (RIA) or enzyme-linked immunoadsorbant assay (ELISA). Such techniques are known in the art and within the skill of the artisan. The binding affinity of the monoclonal antibody to the target can, for example, be determined by a Scatchard analysis (Munson et al., Anal. Biochem., 107:220 (1980)).

After the desired hybridoma cells are identified, the clones may be subcloned by limiting dilution procedures and grown by standard methods (Goding, supra). Suitable culture media for this purpose include, for example, Dulbecco's Modified Eagle's Medium and RPMI-1640. The monoclonal antibodies secreted by the subclones may be isolated or purified from the culture medium by conventional immunoglobulin purification procedures such as, e.g., protein A-sepharose, hydroxyspatite chromatography, gel exclusion chromatography, gel electrophoresis, dialysis, or affinity chromatography.

A variety of methods exist in the art for the production of monoclonal antibodies and thus, the invention is not limited to their sole production in hybridomas. For example, the monoclonal antibodies may be made by recombinant DNA methods, such as those described in U.S. Pat. No. 4,816,567. In this context, the term “monoclonal antibody’ refers to an antibody derived from a single eukaryotic, phage, or prokaryotic clone. The DNA encoding the monoclonal antibodies of the invention can be readily isolated and sequenced using conventional procedures (e.g., by using oligonucleotide probes that are capable of binding specifically to genes encoding the heavy and light chains of marine antibodies, or such chains from human, humanized, or other sources). The hydridoma cells of the invention serve as a preferred source of such DNA. Once isolated, the DNA may be placed into expression vectors, which are then transformed into host cells such as NS0 cells, Simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al, supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain cross-linking. Alternatively, the relevant cysteine residues are substituted with another amino acid residue or are deleted so as to prevent cross-linking.

Antibody fragments which recognize specific epitopes may be generated by known techniques. For example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

For some uses, including in vivo use of antibodies in humans, it may be preferable to use chimeric, humanized, or human antibodies. A chimeric antibody is a molecule in which different portions of the antibody are derived from different animal species, such as antibodies having a variable region derived from a marine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816,397, which are incorporated herein by reference in their entirety.

Humanized antibodies are antibody molecules generated in a non-human species that bind the desired antigen having one or more complementarily determining regions (CDRs) from the non-human species and framework (FR) regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties). Antibodies can be humanized using a variety of techniques known in the art including, for example, CDR-grafting (EP 239, 400; POT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska, et al., PNAS 91:969-973 (1994)), and chain shuffling (U.S. Pat. No. 5,565,332).

Generally, a humanized antibody has one or more amino acid residues introduced into it from a source that is non-human. These non-human amino acid residues are often referred to as “import” residues, which are typically taken from an “import” variable domain. Humanization can be essentially performed following the methods of Winter and co-workers (Jones et al., Nature, 321:522-525 (1986); Reichmann et al., Nature, 332:323-327 (1988); Verhoeyen et al., Science, 239:1534-1536 (1988), by substituting rodent CDRs or CDR sequences for the corresponding sequences of a human antibody.

Accordingly, such “humanized” antibodies are chimeric antibodies (U.S. Pat. No. 4,816,567) herein substantially less than an intact human variable domain has been substituted by the corresponding sequence from a non-human species. In practice, humanized antibodies are typically human antibodies in which some CDR residues and possible some FR residues are substituted from analogous sites in rodent antibodies.

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111 and PCT publications WO 98/46645, WO 98/50433, WO 98/24893, WO 98/16654, WO 96/34096, WO 96/33735, and WO 91/10741; each of which is incorporated herein by reference in its entirety. The techniques of Cole et al., and Boerder et al., are also available for the preparation of human monoclonal antibodies (Cole et al., Monoclonal Antibodies and Cancer Therapy, Alan R. Riss, (1985); and Boerner et al., J. Immunol., 147(1):86-95, (1991)).

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.), (Genpharm (San Jose, Calif.), and Medarex, Inc. (Princeton, N.J.) can be engaged to provide human antibodies directed against a selected antigen using technology similar to that described above.

Also human MAbs could be made by immunizing mice transplanted with human peripheral blood leukocytes, splenocytes or bone marrows (e.g., Trioma techniques of XTL). Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Further, antibodies to the polypeptides of the invention can, in turn, be utilized to generate anti-idiotype antibodies that “mimic” polypeptides of the invention using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide of the invention and/or to bind its ligands/receptors, and thereby block its biological activity.

The antibodies of the present invention may be bispecific antibodies. Bispecific antibodies are monoclonal, preferably human or humanized, antibodies that have binding specificities for at least two different antigens. In the present invention, one of the binding specificities may be directed towards the target, the other may be for any other antigen, and preferably for a cell-surface protein, receptor, receptor subunit, tissue-specific antigen, virally derived protein, virally encoded envelope protein, bacterially derived protein, or bacterial surface protein, etc.

Methods for making bispecific antibodies are well known. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It may have the first heavy-chain constant region (CH1) containing the site necessary for light-chain binding present in at least one of the fusions. DNAs encoding the immunoglobulin heavy chain fusions and, if desired, the immunoglobulin light chain, are inserted into separate expression vectors, and are co-transformed into a suitable host organism. For further details of generating bispecific antibodies see, for example Suresh et al., Meth. In Enzym., 121:210 (1986).

In addition, one can generate single-domain antibodies to the target. Examples of this technology have been described in WO9425591 for antibodies derived from Camelidae heavy chain Ig, as well in US20030130496 describing the isolation of single domain fully human antibodies from phage libraries.

### EXAMPLES

This invention can be further illustrated by the following examples of preferred embodiments thereof, although it will be understood that these examples are included merely for purposes of illustration and are not intended to limit the scope of the invention unless otherwise specifically indicated.

### Example 1

### Recombinant Virus Assay for Determining In Vitro Susceptibility to Drug

Treatment of HIV-infected patients with a first entry inhibitor results in viruses that exhibit increased resistance to the first inhibitor and increased susceptibility to a second entry inhibitor. This was demonstrated by isolating 5A8 resistant viruses from HIV-infected patients treated with 5A8 and testing the susceptibility of those viruses to sCD4.

Viruses to be tested were isolated from subjects which received the entry inhibitor 5A8. In this trial, 5A8 was administered as a single new drug to 22 HIV-1 infected subjects that had stable baseline viral loads of >5000 copies/mL and CD4+ cell counts >100/L. Subjects were randomized among 3 cohorts according to the following schedule:

| | Cohort A | Cohort B | Cohort C |
|---|---|---|---|
| [5A8] | 25 mg/kg | 10 mg/kg | 25 mg/kg |
| | | first dose; | |
| | | 6 mg/kg | |
| | | subsequent doses | |
| Frequency | Once every 7 | Once every 14 | Once every 14 |
| | days for 9 weeks | days for 9 weeks | days for 8 weeks |

At each visit when 5A8 was administered, two blood samples were collected from each subject, one within one hour prior to infusion and one at the completion of the infusion.

A recombinant virus assay was used to determine the in vitro susceptibility of viral isolates to a second entry inhibitor. Libraries of HIV viral genomic RNA were generated from HIV present in the sera of 4 of the 22 subjects before therapy (baseline) and 9 weeks after therapy with 5A8 for the analysis. Viral genomic RNA was isolated from the subjects' sera by using oligo(dT) magnetic beads.

First-strand cDNA was synthesized in a standard reverse transcription reaction by using commercially available oligo(dT) primer. Envelope DNA (“ENV”) encoding the viral protein gp160 (comprising gp120 and gp41) was amplified by PCR using forward and reverse primers located immediately upstream and downstream of the ENV initiation and termination codons, respectively. The forward and reverse primers (see Table 3 of U.S. Application No. 2005/0214743 (incorporated herein by reference)) contain recognition sites for PinAI and MluI, respectively. Env PCR products were digested with PinAI and MluI and ligated to compatible ends in a pCXAS expression vector, which uses the cytomegalovirus immediate-early promoter enhancer to drive ENV insert expression in transfected cells. The construction of the pCXAS vectors has been described in U.S. Pat. No. 5,837,464. Ligation products were introduced into competent Escherichia coli (Invitrogen) by transformation, and pCXAS-ENV plasmid DNA was purified from bacterial cultures (Qiagen, Valencia, Calif.).

An aliquot of each transformation was plated onto agar, and colony counts were used to estimate the number of envelope sequences represented in each pCXAS-ENV library (generally 500-5,000 clones). Sequence analysis of individual pCXAS-ENV clones (10-20) was used to verify the heterogeneous composition (i.e., quasispecies) of pCXAS-ENV libraries. 27 individual subclones from baseline and 9 week samples were used in the studies. Pseudotyped HIV particles containing envelope proteins encoded by the subject-derived segment were produced by transfecting a packaging host cell (HEK 293) with resistance test vector DNA. Virus particles were collected (48 h) after transfection and were used to infect target cells (HT4/CCR5/CXCR4, or U-87/CD4/CXCR4, or U-87/CD4/CCR5) that express HIV receptors (i.e. CD4) and co-receptors (i.e. CXCR4, CCR5). After infection (^{˜}72 h) the target cells were lysed and luciferase activity was measured. HIV must complete one round of replication to successfully infect the target host cell and produce luciferase activity. The amount of luciferase activity detected in the infected cells is used as a direct measure of “infectivity”. If for any reason (e.g., lack of the appropriate receptor or co-receptor, inhibitory drug activity, neutralizing antibody binding), the virus is unable to enter the target cell, luciferase activity is diminished. Drug susceptibility is assessed by comparing the infectivity in the absence of drug to infectivity in the presence of drug. Relative drug susceptibility can be quantified by comparing the susceptibility of the “test” virus to the susceptibility of a well-characterized reference virus (wildtype) derived from a molecular clone of HIV-1, for example NL4-3 or HXB2.

Packaging host cells were seeded in 10 cm-diameter dishes and were transfected one day after plating with pHIVenv and pHIVluc. Transfections were performed using a calcium-phosphate co-precipitation procedure. The cell culture media containing the DNA precipitate was replaced with fresh medium, from one to 24 hours, after transfection. Cell culture media containing viral particles was typically harvested 2 days after transfection and was passed through a 0.45-mm filter. Before infection, target cells were plated in cell culture media. Entry inhibitor drugs were typically added to target cells at the time of infection (one day prior to infection on occasion). Typically, 3 days after infection target cells were assayed for luciferase activity using the Steady-Glo agent (Promega) and a luminometer.

Activity is represented as i) the inhibitor concentration conferring 50% inhibition (IC50) or ii) the mean percent inhibition of the 3 highest inhibitor concentrations (percent maximal inhibition, PMI). The results are presented in the tables below. Viral isolates showed a range of susceptibility to 5A8 (an anti-CD4 MAb), and can be grouped into three categories: i) highly susceptible to 5A8 (Table 1), ii) intermediately resistant to 5A8 (Table 2) or iii) highly resistant to 5A8 (Table 3). The highly susceptible group exhibited a range of 5A8 PMI from 87% to 100%. The intermediately resistant isolates exhibited a range of 5A8 PMI of 33.3% to 64.7%. 5A8 PMI in the highly resistant isolates ranged from below zero to 22.8%.

The data also show that susceptibility to soluble CD4 (sCD4) was inversely correlated with susceptibility to 5A8. The mean sCD4 IC50 for the highly susceptible isolates, the intermediately resistant isolates and the highly resistant isolates were 25.72 ug/mL, 10.86 ug/mL and 4.25 ug/mL, respectively.

TABLE 1
Activity of sCD4 against viruses highly susceptible to 5A8

| | | sCD4 IC₅₀ | Mean sCD4 |
|---|---|---|---|
| Virus Sublcone | 5A8 PMI (%) | (ug/mL) | IC₅₀ (ug/mL) |
| E03_4338_32 | 87.0 | 50.00 | 25.72 |
| E03_4330_03 | 100.0 | 50.00 | |
| E03_4328_01 | 100.0 | 50.00 | |
| E03_4328_09 | 96.6 | 29.95 | |
| E03_4331_03 | 98.2 | 19.47 | |
| E03_4331_09 | 97.2 | 17.48 | |
| E03_4330_05 | 100.0 | 10.32 | |
| E03_4333_06 | 98.0 | 3.20 | |
| E03_4333_05 | 97.7 | 1.07 | |

TABLE 2
Activity of sCD4 Against Viruses with Intermediate
Levels of Resistance to 5A8

| | | sCD4 IC₅₀ | Mean sCD4 |
|---|---|---|---|
| Virus Sublcone | 5A8 PMI (%) | (ug/mL) | IC₅₀ (ug/mL) |
| E03_4328_34 | 39.7 | 50.00 | 10.86 |
| E03_4340_48 | 62.4 | 26.47 | |
| E03_4341_04 | 42.0 | 19.01 | |
| E03_4338_25 | 42.3 | 16.44 | |
| E03_4343_15 | 37.5 | 11.00 | |
| E03_4343_02 | 35.4 | 6.06 | |
| E03_4340_02 | 61.4 | 4.04 | |
| E03_4343_06 | 33.3 | 2.99 | |
| E03_4343_04 | 57.9 | 2.55 | |
| E03_4328_11 | 48.6 | 1.12 | |
| E03_4340_09 | 64.7 | 0.72 | |
| E03_4340_30 | 47.1 | 0.41 | |
| E03_4341_09 | 33.6 | 0.39 | |

TABLE 3
Activity of sCD4 Against Viruses Highly Resistant to 5A8

| | | sCD4 IC₅₀ | Mean sCD4 |
|---|---|---|---|
| Virus Sublcone | 5A8 PMI (%) | (ug/mL) | IC₅₀ (ug/mL) |
| E03_4338_10 | −24.1 | 18.65 | 4.25 |
| E03_4341_05 | 17.4 | 1.65 | |
| E03_4341_42 | 17.6 | 0.60 | |
| E03_4341_15 | 22.8 | 0.20 | |
| E03_4338_26 | 5.7 | 0.14 | |

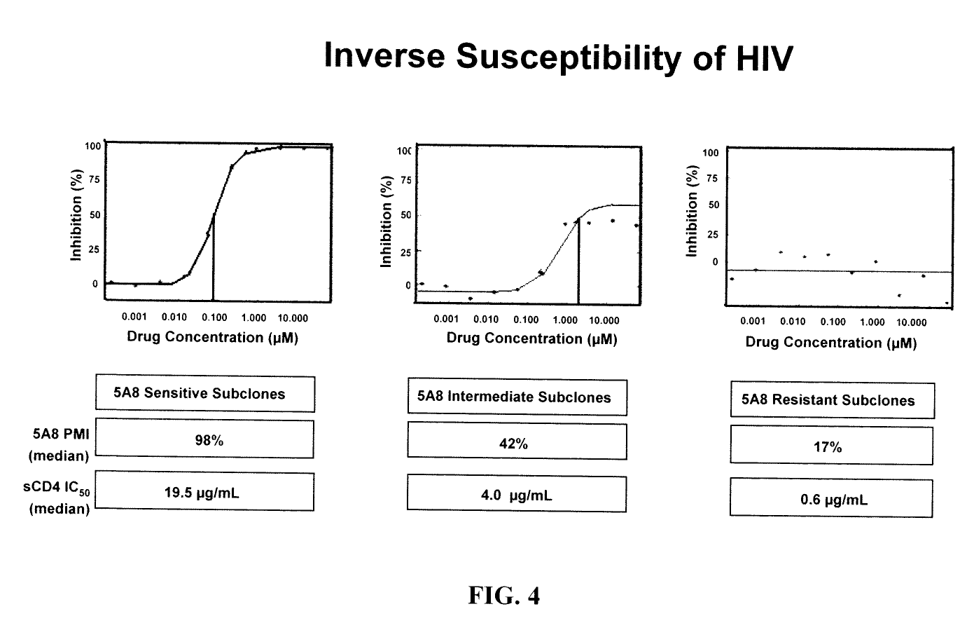

Referring to Tables 1, 2, and 3, the data show that treatment with the first entry inhibitor 5A8 produced resistant HIV that was highly susceptible to neutralization by a second HIV entry inhibitor, soluble CD4. This data is graphically depicted in FIG. 4.

### Example 2

### Analysis of sCD4-Fc Variants by ELISA

Variants of sCD4 can be tested for their ability to bind HIV according to the following assay. A mouse monoclonal antibody (Sim-2) was used to detect binding of sCD4 variants. SIM2 is diluted in PBS to a final concentration of 0.2 μg/ml and coated on 96-well plates at 100 μl per well. The plates are incubated overnight at room temperature to ensure attachment of the antibody to the plate. The solution is then removed and the plates are washed two to three times with phosphate buffered saline containing TWEEN® (PBST).

Non-specific binding sites are blocked using 200 μl of 2% BSA/PBST, incubating for 30 min at room temperature. Wells are then washed twice with PBST. Variants to be tested are added to the plate at 100 μl/well. Each sample is done in duplicate. Appropriate negative controls are provided using either buffer or an irrelevant peptide that is not recognized by SIM2. Samples are incubated at room temperature for 1-2 hr, followed by removal of the contents of the well and washing two to three times with PBST.

The bound variant sCD4 is detected by adding a secondary detection antibody at a dilution of 1:2000 in 2% BSA/PBST. 100 μl of diluted HRP-conjugated goat anti-human IgG Fc antibody is added and the plate is incubated at room temperature for 1-2 hr. The wells are then washed 5 times with PBST. HRP is detected by adding 100 μl of TMB substrate solution to each well followed by incubation at room temperature for 5-30 min. Positives appear pale blue. 50 μl of 1.0M H₂SO₄ is added to each well to stop the reaction. Positives now appear bright yellow. Plates are read at 450 nm.

### Example 3

### Analysis of Binding of sCD4-Fc Variants to gp120 by FACS

In this experiment gp120-transfected HeLa cells are resuspended at a concentration of 1×106 HL2/3 cells in 50 μl of ice-cold FACS buffer. Cells are divided into two tubes at 25 μl per tube. Testing samples are added at a volume of 25 μl to one tube and 25 μl of an irrelevant Fc-fusion protein to the other tube (served as negative control). Samples are incubated on ice for 10-30 min.

Following the incubation, cells are pelleted and washed with 0.5 ml FACS buffer. Cells are then resuspended in 25 μl of FACS buffer and mixed with 25 μl of diluted R-Phycoerythrin (R-PE)-conjugated anti-human IgG Fe antibody. The samples are incubated on ice for another 10-30 min followed by centrifugation and two washes using 0.5 ml FACS buffer. Cells are resuspended in 0.5 ml FACS buffer and analyzed using flow cytometry. The amount of sCD4-Fc of a particular variant bound to the gp120 expressing cell is measured as a function of fluorescence intensity. Cells can also be fixed for later analysis. To fix cells, resuspend cells in 0.5 ml of 1% paraformaldehyde/PBS.

### Example 4

### Construction of T-20-Resistant HIV Envelope Expression Vectors

A DNA expression vector comprising the envelope protein of HIV strain JRCSF was constructed using a commercially available pCI-neo vector (Promega, Madison, Wis.). JRCSF is a primary isolate obtained from an HIV patient which was deposited with the NIH AIDS Research & Reference Reagent Program (Cat. No. 2708). The JRCSF envelope open reading frame was amplified by standard PCR using the following primers:

(SEQ ID NO 4)

5′-GATCGAATTCACGCGTAGCAGAAGACAGTGGCAATGA-3′
and

(SEQ ID NO 5)

5′-GATCGTCGACTCTAGATTTTGACCACTTGCCACCCAT-3′.

These primers initiate priming at codon 2 of the envelope open reading frame and immediately downstream of the envelope termination codon, respectively. They contain restriction enzymes recognition sequences suitable for directional cloning into the pCI-neo vector. This JRCSF envelope protein expression vector was used in Example 6 below as the WT control.

In addition, three single amino acid substitutions, G36D, V38A and N43D, known to confer T-20 resistance for HIV were introduced into the JRCSF open reading frame by site-directed mutagenesis (QuikChange II kit, Stratagene, La Jolla, Calif.), thereby generating three T-20-resistant HIV envelope protein expression vectors called G3D, V38A and N43D.

### Example 5

### Patient-Derived HIV Envelope Protein Expression Vectors

HIV RNA was extracted from patient serum using QIAamp isolation kits (Qiagen, Hilden. Germany) and used to generate cDNA with random hexamer primers (SuperScript First Strand Synthesis System for RT-PCR, Invitrogen, Carlsbad, Calif.). HIV envelope protein open reading frame sequences were specifically amplified using a nested PCR procedure with outer PCR primers:

| | |
|---|---|
| 5′-GGCTTAGGCATCTCCTATGGCAGGAAGAA-3′ | (SEQ ID NO 6) |
| and | |
| 5′-CTGCCAATCAGGGAAGTAGCCTTGTGT-3′ | (SEQ ID NO 7) |

which initiate priming 239 bases upstream and 349 bases downstream of the envelope open reading frame, respectively. The inner PCR primers were the same primers used for directional cloning of the envelope sequence populations into the pCI-neo vector, as described in Example 4. The resulting PCR products represent a population of envelope sequences from HIV viruses present in two patient samples, KJM (day 1) and KJM (week 9).

### Example 6

### Preparation of Pseudotyped HIV Reporter Viruses

HIV reporter viruses for purposes of this experiment were constructed from: (1) the JRCSF WT envelope protein vector, (2) the T-20-resistant envelope protein variants G36D, V38A, or N43D generated in Example 4; or (3) the patient derived HIV envelope protein population of vectors from KJM (Day 1) or KJM (Week9) generated in Example 5. The reporter viruses were produced by co-transfecting the human embryonic kidney cell line HEK293FT (Invitrogen, Carlsbad, Calif.) with the envelope vectors described above and an envelope-deficient proviral DNA encoding a luciferase reporter gene. The cells were propagated in Dulbecco's Modified Eagle Medium (Invitrogen, Carlsbad, Calif.) supplemented with 10% fetal bovine serum (Invitrogen, Carlsbad, Calif.). Transfections were performed using Lipofectamine 2000 (Invitrogen, Carlsbad, Calif.). Culture supernatants were harvested 48 hours post-transfection and filtered through a 0.45 micron filter prior to infecting target cells. This transient transfection resulted in the generation of new viral particles comprising the luciferase reporter gene and an envelope protein derived from one of the envelope protein vectors described above.

### Example 7

### Single-Cycle Infection and Dose-Response

Viral infections were carried out in a human malignant glioma (U87) target cell expressing the human CD4 receptor and the human CCR5 chemokine receptor. Target cells were seeded into 96-well plates at a density of 3000 cells/well in Dulbecco's Modified Eagle Medium (Invitrogen, Carlsbad, Calif.) supplemented with 10% fetal bovine serum (Invitrogen. Carlsbad, Calif.) and cultured for 18-24. Target cells were then exposed to the virus generated in Example 6 mixed with varying concentrations of the entry inhibitors T-20 or an anti-CD4 MAb in a final volume of 40-80 microliters. Infection was triggered by centrifugation of the 96-well plates at 1200*g for 1.5-2.0 hours at 25° C. (i.e. spinoculation). After spinoculation, culture medium was added to a final volume of 80 microliters per well and the infected cultures were incubated for 3 days at 37° C. After 3 days the level of luciferase expressed in target cells was measured using a luciferase reporter gene assay reagent (Promega, Madison, Wis.), as described by the manufacturer.

### Example 8

### EC₅₀ Determinations

Luciferase levels measured in the presence of the entry inhibitor 5A8 (an anti-CD4 MAb) or T-20 were used to calculate the fraction of luciferase remaining as compared to the luciferase level in cells infected in the absence of entry inhibitor. Dose-response curves for the inhibition of luciferase expression by entry inhibitors were derived by fitting the luciferase data with a four-parameter logistic using Origin 7 Client software (OriginLab, Northampton, Mass.). Each dose-response experiment comprised a nine point concentration curve plus untreated controls, all assayed in triplicate wells (raw data not shown). For each EC50 determination, the results of three or more independent experiments were used to calculate the average EC₅₀ and standard error values.

In Vitro Susceptibility of T-20 Resistant HIV

| | EC50, ng/ml^{a} | | Fold Change^{b} | | PMI | |
|---|---|---|---|---|---|---|
| | 5A8 | T-20 | 5A8 | T-20 | 5A8 | T-20 |
| JRCSF | 18 ± 8.1 | 13 ± 3.5 | — | — | 95 ± 3.4 | 98 ± 2.4 |
| G36D | 37 ± 13 | 140 ± 26 | 2.0 | 11 | 92 ± 1.6 | 99 ± 1.5 |
| V38A | 24 ± 5.2 | 410 ± 210 | 1.3 | 32 | 97 ± 0.82 | 91 ± 8.9 |
| N43D | 20 ± 11 | 280 ± 80 | 1.1 | 22 | 95 ± 2.6 | 96 ± 3.4 |

^{a}Average +/− standard error from 4 independent determinations
^{b}EC50_{MUT}/EC50_{WT} JRCSF

In Vitro Susceptibility of 5A8 Resistant HIV

| | EC50, ng/ml^{a} | | Fold Change^{b} | | PMI | |
|---|---|---|---|---|---|---|
| | 5A8 | T-20 | 5A8 | T-20 | 5A8 | T-20 |
| KJM D1 | 15 ± 8.7 | 9.1 ± 3.0 | — | — | 97 ± 1.6 | 99 ± 1.3 |
| KJM W9 | 28 ± 9.4 | 8.5 ± 2.7 | 1.9 | 0.93 | 73 ± 12 | 100 ± 0.58 |

^{a}Average +/− standard error from 4 independent determinations
^{b}EC50_{W9}/EC50_{D1} JRCSF

In the specification, there have been disclosed typical embodiments of the invention and, although specific terms are employed, they are used in a generic and descriptive sense only and not for purposes of limitation.

1. A method for inhibiting HIV infection in a subject in need thereof comprising administering a first HIV entry inhibitor, and upon emergence of resistant HIV, administering a second entry inhibitor.

2. The method of claim 1 wherein the first entry inhibitor: (1) inhibits HIV entry by binding CD4 receptors; (2) is a co-receptor inhibitor; or (3) is a fusion inhibitor.

3. The method of claim 2, wherein the first entry inhibitor is an anti-CXCR4 or an anti-CCR5 antibody.

4. The method of claim 2, wherein the first entry inhibitor is BMS-806, Sch-D, GW-873,140, UK-427,857, PRO-140, AMD-070, or T-20.

5. The method of claim 2 wherein the first entry inhibitor is an anti-CD4 antibody.

6. The method of claim 5, wherein the anti-CD4 antibody comprises SEQ ID NO 1 and SEQ ID NO 2.

7. The method of claim 5, wherein the anti-CD4 antibody comprises the following CDRs: (a) light chain CDR1 comprising AA24-AA40 of SEQ ID NO: 2; (b) light chain CDR2 comprising AA56-AA62 of SEQ ID NO: 2; (c) light chain CDR3 comprising AA95-AA102 of SEQ ID NO: 2, (d) heavy chain CDR1 comprising AA31-AA35 of SEQ ID NO: 1; (e) heavy chain CDR2 comprising AA50-AA66 of SEQ ID NO: 1; and (f) heavy chain CDR3 comprising AA99-AA111 of SEQ ID NO: 1.

8. The method of claim 5 wherein the antibody is an anti-CD4 antibody that permits the binding of gp120 to the CD4 receptor but inhibits HIV entry.

9. The method of claim 1, wherein the second entry inhibitor is an HIV gp120 inhibitor.

10. The method of claim 9, wherein the second entry inhibitor is sCD4 or its variants or PRO-542 or an anti-gp120 antibody.

11. The method of claim 1, wherein the second entry inhibitor is a fusion inhibitor.

12. The method of claim 11, wherein the fusion inhibitor is T-20.

13. The method of claim 1, further comprising administering at least one non-entry inhibitor anti-HIV drug.

14. The method of claim 13, wherein the non-entry inhibitor is an integrase inhibitor, a nucleoside reverse transcriptase inhibitor, a non-nucleoside reverse transcriptase inhibitor, or an HIV protease inhibitor.

15. A method for overcoming HIV drug resistance due to the treatment with a first entry inhibitor in a subject having an HIV infection comprising administering a second HIV entry inhibitor different from the first entry inhibitor.

16. The method of claim 15, wherein drug resistant HIV released from treated infected cells exhibit an altered phenotype relative to non-resistant HIV, wherein said altered phenotype renders the resistant HIV susceptible to a second HIV entry inhibitor.

17. A method to increase CD4+ T lymphocyte immune responsiveness in a patient infected with HIV, comprising administering a first HIV entry inhibitor and upon emergence of resistant HIV, administering a second entry inhibitor.

18. The method of claim 1, 9, 13, 15 or 17, wherein the first entry inhibitor and the second entry inhibitor are administered simultaneously or sequentially.

19. A composition comprising an admixture of at least two HIV entry inhibitors, wherein one entry inhibitor selects for a resistant HIV that are highly susceptible to neutralization by the other HIV entry inhibitor.

20. The composition of claim 19, wherein one entry inhibitor blocks HIV-1 by binding CD4 receptor.

21. The composition of claim 20, wherein the entry inhibitor is an anti-CD4 antibody or a binding fragment thereof.

22. The composition of claim 19, wherein the other entry inhibitor is sCD4 or a variant thereof, a CD4-immunoglobulin fusion protein, such as CD4-IgG2, or an entry inhibitor that binds HIV-1 gp120 envelope glycoproteins, such as an anti-gp120 antibody.

23. The composition of claim 19, wherein at least one entry inhibitor is an anti-CCR5 or anti-CXCR4 antibody.

24. The composition of claim 19, wherein at least one entry inhibitor is a fusion inhibitor, such as T-20.

25. The composition of claim 21 or 23, wherein the antibody is a monoclonal antibody.

26. The composition of claim 25, wherein the monoclonal antibody is a human, humanized or chimeric antibody.

27. The composition of claim 25, wherein the antibody is a Fab fragment.

28. The composition of claim 27, wherein the Fab fragment comprises the variable domain of the antibody.

29. The composition of claim 27, wherein the antibody Fab fragment comprises a CDR region of the antibody.

30. A kit comprising a composition comprising at least two HIV entry inhibitors, wherein one entry inhibitor selects for a resistant HIV that are highly susceptible to neutralization by the other HIV entry inhibitor.

31. The kit of claim 30 comprising two vials, one vial containing one entry inhibitor and a second vial containing the other entry inhibitor.

32. The kit of claim 30, comprising in separate containers in a single package a combination of two or more different entry inhibitors.

33. The kit of claim 32, further comprising at least one non-entry inhibitor anti-HIV drug useful for inhibiting or preventing HIV infection of target cells or for the prevention or treatment of HIV infection.

34. The kit of claim 33, wherein at least one non-entry inhibitor anti-HIV drug is an integrase inhibitor, a nucleoside reverse transcriptase inhibitor, a non-nucleoside reverse transcriptase inhibitor, or a protease inhibitor.
